(19) United States
(12) Patent Application Publication
(10) Pub. No.: US 2025/0306153 A1
(43) Pub. Date: Oct. 2, 2025

(54) Elastography Apparatus for MR Elastography of a Head

(71) Applicants: Siemens Healthineers AG, Forchheim (DE); Centre National de la Recherche Scientifique (CNRS), Paris Cedex 16 (FR); Institut National de La Sante et de la Recherche Medicale (INSERM), Paris (FR); King's College London, London (GB); Université Paris XIII Paris-Nord, Villetaneuse (FR); Université Paris Cité, Paris (FR)

(72) Inventors: Katharina Schregel, Dossenheim (DE); Omar Darwish, London (GB); Gustav Neitz, Dossenheim (DE); Giacomo Annio, Mola Di Bari Ba (IT); Ralph Sinkus, Parmain (FR)

(73) Assignees: Siemens Healthineers AG, Forchheim (DE); Centre National de la Recherche Scientifique (CNRS), Paris Cedex 16 (FR); Institut National de La Sante et de la Recherche Medicale (INSERM), Paris (FR); King's College London, London (GB); Université Paris XIII Paris-Nord, Villetaneuse (FR); Université Paris Cité, Paris (FR)

(21) Appl. No.: 19/091,955

(22) Filed: Mar. 27, 2025

(30) Foreign Application Priority Data
Mar. 28, 2024 (EP) 24167480.3

(51) Int. Cl. G01R 33/563; A61B 5/00; A61B 5/055

(52) U.S. Cl. CPC G01R 33/56358; A61B 5/0042; A61B 5/0051; A61B 5/055; A61B 2560/0443

(57) ABSTRACT

An elastography apparatus is described that is designed to assist in magnetic resonance elastography of the head of an examination object. The elastography apparatus comprises a first support element which is designed to be positioned within a radiofrequency receiving coil unit and a vibration generator which is designed to generate mechanical waves. The first support element has a first recess for receiving the vibration generator, the first support element has a second recess for receiving the head, and the first recess is so shaped as to at least partially enclose the vibration generator in a custom-fit manner and the vibration generator is arranged at least partially within the first recess.

### CROSS-REFERENCE TO RELATED APPLICATIONS

The present application claims priority to and the benefit of European patent application no. EP 24167480.3), filed on Mar. 28, 2024, the contents of which are incorporated herein by reference in their entirety.

### TECHNICAL FIELD

The disclosure relates to an elastography apparatus for a magnetic resonance device, a receiving unit comprising a radiofrequency (RF) receiving coil, an elastography apparatus for use in combination with a magnetic resonance device, and a magnetic resonance device.

### BACKGROUND

In a magnetic resonance device, the body of an examination object, e.g. a patient, that is to be examined is usually exposed to a relatively strong main magnetic field, for example 1.5 or 3 tesla, by means of a main magnet. In the context of magnetic resonance imaging (MR imaging), gradient pulses are emitted with the aid of a gradient coil unit. In addition, high-frequency radiofrequency pulses (RF pulses), e.g. excitation pulses, are then transmitted via a radiofrequency (RF) antenna unit by means of suitable antenna devices, whereby the nuclear spins of specific atoms that are resonantly excited by these RF pulses are tilted about a defined flip angle relative to the magnetic field lines of the main magnetic field. During the relaxation of the nuclear spins, radiofrequency signals (so-called magnetic resonance signals) are emitted, to be received by means of suitable radiofrequency antennas and then processed further. The desired image data can subsequently be reconstructed from the raw data acquired thus. For a specific measurement, it is necessary to transmit a specific magnetic resonance control sequence (MR control sequence) consisting of a sequence of radiofrequency pulses, for example excitation pulses and refocusing pulses, as well as corresponding gradient pulses that are transmitted in a coordinated manner in various gradient axes along various spatial directions. Temporally corresponding readout windows are set, and specify the time periods during which the induced magnetic resonance signals are captured.

The radiofrequency antennas for receiving the magnetic resonance signals are typically part of a radiofrequency receiving coil unit, and are arranged as close as possible to that region of the examination object which is to be examined. Dedicated radiofrequency receiving coil units are available for various regions of the body that are to be examined.

In the case of MR elastography (MRE), the fact is exploited that the phase of the magnetic resonance signals changes as a result of mechanical waves that act on the examination object. The extent of this change depends on the deflection (i.e. the displacement from the position of rest) of the tissue as a result of the mechanical waves. This means that the MR phase images, i.e. images which represent the phase of the nuclear magnetization, can be used to derive information relating to specific mechanical parameters of the tissue, for example the elasticity. MRE is therefore a non-invasive method for quantifying the elasticity and stiffness of a tissue. In addition to a conventional magnetic resonance device, MRE requires a vibration generator for generating the mechanical waves, such as in the examination region of the examination object.

With regard to the head in particular, changes in the consistency of the tissue can indicate neurological diseases and/or a pathology. Biomechanical information relating to brain tumors is also valuable for the planning of surgical interventions, tumor characterization and treatment monitoring. Until now, MRE in the region of the head has only been carried out in the experimental environment, particularly with a view to acquisition methods, vibration frequency, and the devices used.

### SUMMARY

The object of the disclosure is to specify a particularly resilient and reliable elastography apparatus for MRE in the head, which is also comfortable for the patient. The object is achieved by the features described in the various embodiments throughout the disclosure, including the claims.

The elastography apparatus, which is designed to assist in magnetic resonance elastography of the head of an examination object, comprises a first support element which is designed to be positioned within a radiofrequency receiving coil unit and a vibration generator which is designed to generate mechanical waves,
the first support element having a first recess for receiving the vibration generator,
the first support element having a second recess for receiving the head, and
the first recess being so shaped as to at least partially enclose the vibration generator in a custom-fit manner and the vibration generator being and/or potentially being arranged at least partially within the first recess.

The elastography apparatus may e.g. be designed to execute a method for MRE in combination with a radiofrequency receiving coil unit and a magnetic resonance device. The radiofrequency receiving coil unit is typically a head coil unit, e.g. a receiving coil unit which is designed to receive MR signals from the head and/or neck of an examination object when the head and/or neck of an examination object is positioned at least partially within the radiofrequency receiving coil unit and within a patient receiving region of a magnetic resonance device. The vibration generator is designed to output mechanical vibrations, e.g. with shear components and compression components. As described in U.S. Pat. No. 7,034,534B2, for example, the vibration generator can have a flexible membrane which is stimulated by means of acoustic energy to produce vibration. The vibration generator can be designed as a gravitational vibration generator.

The first support element can be designed as a support surface and/or locating unit which is for a head of an examination object and is to be positioned within a head coil unit. The first support element can comprise plastic and/or foam, for example. The first recess and the second recess may e.g. be arranged on a first side, e.g. on the upper side of the first support element. The opposite side, e.g. the lower side, can be designed in accordance with the shape of the radiofrequency receiving coil unit. Likewise, the shape of the first support element can be designed in accordance with the shape of the radiofrequency receiving coil unit, for example as an oval.

The first recess is typically designed as a hollow of the first support element. The first recess may e.g. be shaped in such a way that the vibration generator can be at least partially enclosed in a custom-fit manner by the first recess and/or the vibration generator is located and/or held in a stable position when positioned at least partially within the first recess. The first recess can therefore serve as a positioning aid and/or locating aid for the vibration generator.

The second recess is typically designed as a hollow of the first support element. The second recess may e.g. be shaped in such a way that it can at least partially accommodate a head, e.g. the back of a head, and/or at least partially accommodate a neck of the examination object. The first recess and the second recess are typically positioned relative to each other in such a way that when the vibration generator is located in the first recess and a head is positioned in the second recess, the vibration generator is designed to initiate mechanical waves in the head. The first recess and the second recess are typically separated by less any suitable distance, such as e.g. less than 6 cm, less than 4 cm, less than 2 cm, etc. The first recess and the second recess can be disjunct. The first recess and the second recess can merge into each other and/or be interconnected. When the vibration generator and the head are arranged on the first support element, the vibration generator, and the head may be separated by less than any suitable distance, such as e.g. less than 4 cm, less than 3 cm, less than 2 cm, etc. When the vibration generator and the head are arranged on the first support element, the vibration generator and the head may have a contact point and/or a contact surface between them. When the vibration generator and the head are arranged on the first support element, a contact element, e.g. a cushion, can be arranged between the vibration generator and the head.

The elastography apparatus allows precise positioning of the vibration generator relative to the head that is to be positioned, whereby effective and reliable contact between the two is possible. In addition, the vibration generator can typically be so positioned as to be independently stable in the first recess, such that its position is protected from any pressure due to the weight of the head. The head can be located at least partially within the second recess, it being possible to preset the position of the head relative to the vibration generator by means of the second recess. This allows for adequate penetration, in the head, of the waves that are generated by the vibration generator. In an embodiment, the transverse waves are free from higher order waves during propagation and exhibit only slight non-linearities. The elastography apparatus therefore allows for resilient and reliable mechanical stimulation for MRE in the head, while the second recess also allows the head to be located comfortably. In addition, the elastography apparatus can be used in combination with commercially available radiofrequency receiving coil units, which can customarily be used as a receiving coil unit for a head, even without MRE. This elastography apparatus can therefore be used as an accessory and consequently in a particularly economical and flexible manner.

An embodiment variant of the elastography apparatus provides for the first recess to be designed in such a way that the vibration generator can be reversibly and removably positioned in the first recess. The vibration generator can therefore be arranged in the first recess in a flexible manner and as required. In an embodiment, for the purpose of positioning the head on the first support element, the vibration generator can be removed and/or positioned at the same time. In addition, according to this embodiment variant, the vibration generator can be withdrawn for the purpose of cleaning and/or maintenance of the elastography apparatus. This allows the elastography apparatus to be used in a particularly flexible manner.

An embodiment variant of the elastography apparatus provides for the vibration generator to be designed as a gravitational vibration generator having an eccentric mass which can rotate about a rotational axis and a shaft which can rotate about a drive axis that is located parallel with the rotational axis, said shaft being designed to drive the eccentric mass, the drive axis and the rotational axis forming a drive plane.

The gravitational vibration generator can be implemented in accordance with any suitable design, such as those described for instance in accordance with US20230305090A1, for example. US20230305090A1 discloses the way in which such a gravitational vibration generator functions. Such a gravitational vibration generator can be used in a particularly resilient manner and is characterized by stability in the field of mechanical wave generation. In addition, such a gravitational vibration generator can be synchronized particularly effectively with the activation of a magnetic resonance device.

An embodiment variant of the elastography apparatus provides for the first support element to be designed as a surface element in a first plane, and for the first recess to be designed in such a way that when the vibration generator is positioned at least partially within the first recess, the drive plane encloses an angle of between 25° and 65° with the first plane.

The surface element can comprise a foam mat which has a height of, for example, between any suitable range of dimensions, such as e.g. between 0.5 cm and 7 cm, between 1 cm and 5 cm, between 1.5 cm and 3 cm, etc. The height may e.g. correspond to the maximum spatial extent of the first support element perpendicular to the first plane. The upper side of the first support element minus the first recess and the second recess, i.e. assuming the first recess and second recess are filled, can correspond to the first plane.

The first plane can be horizontally oriented. The first recess and the second recess may e.g. be arranged on a first side, e.g. on the upper side of the first support element. The opposite side of the first support element, e.g. the lower side, can be shaped and/or designed in accordance with the shape of the radiofrequency receiving coil unit. Likewise, the shape of the first support element can be designed in accordance with the shape of the radiofrequency receiving coil unit, for example as an oval.

The drive plane of the vibration generator, when the vibration generator is positioned in the first recess according to this embodiment variant, encloses an angle of any suitable range, such as e.g. between 25° and 65°, between 35° and 55°, between 40° and 50°, etc., with the first plane. The drive plane of the vibration generator, when the vibration generator is positioned in the first recess according to this embodiment variant, can enclose an angle of between 43° and 47°, e.g. 45°, with the first plane. The first recess allows such an orientation of the vibration generator by virtue of its shape. Locating the vibration generator in this way allows the mechanical waves to be transmitted in three spatial directions and adequate penetration in the region of the head.

An embodiment variant of the elastography apparatus provides for the drive axis to be closer to the first plane than the rotational axis. According to this embodiment variant, the rotational axis and therefore the eccentric mass likewise are further away from the first plane than the drive axis. This allows the eccentric mass to be positioned particularly close to a head that is located in the second recess, thereby ensuring homogeneous penetration of the head by mechanical waves. In addition, the rotatable shaft is arranged in the region of the first plane, e.g. if the elastography apparatus and/or the first plane is located horizontally. A flexible rotating lead which is used to activate the vibration generator and is connected to the rotatable shaft can therefore be arranged on the first support element and/or partially integrated in the first support element, and can be guided away from the examination region in an ergonomic manner allowing for the anatomy and/or shoulders.

An embodiment variant of the elastography apparatus provides for the first support element to be designed as a surface element in a first plane and for the vibration generator to comprise a housing unit. The housing unit externally seals the components that are included in the gravitational vibration generator, and has at least one flat surface. When the vibration generator is positioned at least partially within the first recess according to this embodiment variant, the flat surface encloses an angle of any suitable range, such as e.g. between 25° and 65°, between 35° and 55°, between 40° and 50°, etc., with the first plane. The flat surface can enclose an angle of between 43° and 57°, e.g. 45°, with the first plane. The flat surface can be parallel with the drive plane. The flat surface can be designed as a plane. The first recess may e.g. be so designed as to be shaped, on that side facing the second recess, at least partially in a custom-fit manner for the flat surface. The housing unit can be designed to be cuboid, e.g. approximately cuboid., excepting for tolerances. The housing unit can have two flat surfaces, the components that are included in the gravitational vibration generator being arranged between the two flat surfaces. The housing unit typically has sides which border the two flat surfaces. The housing unit (also referred to herein as a housing assembly) can have rounded corners and/or rounded edges. The two flat surfaces can be embodied parallel with each other and/or parallel with the drive plane. This embodiment variant allows the vibration generator to be arranged at the head in an anatomical manner, irrespective of the shape of the housing of the vibration generator, and allows a homogeneous penetration of the head by mechanical waves.

An embodiment variant of the elastography apparatus provides for the first support element to be designed as a surface element in a first plane, and for the first recess to be designed in such a way that when the vibration generator is positioned at least partially within the first recess, the drive axis and/or the rotational axis forms any suitable angle (e.g. between 2° and 20°) with the first plane. The rotational axis of the vibration generator, when positioned in the first recess according to this embodiment variant, encloses an angle of any suitable range, such as e.g. between 2° and 20°, between 3° and 10°, between 4° and 8°, etc., with the first plane. The rotational axis of the vibration generator can, when positioned in the first recess according to this embodiment variant, enclose an angle of any suitable range, such as e.g. between 4.5° and 6°, 5°, etc., with the first plane. This embodiment variant therefore provides for the vibration generator to be positioned within the first recess in such a way that the drive axis and the rotational axis do not extend parallel with the first plane. This embodiment variant provides for the rotational axis to be tilted relative to the first plane. Owing to the anatomy of a head which is positioned in the second recess, this allows for good penetration of the mechanical waves in the head, e.g. as a result of a potentially particularly large contact surface between head and vibration generator.

An embodiment variant of the elastography apparatus provides for the first recess to be designed in such a way that when the vibration generator is positioned at least partially within the first recess, the drive axis and/or the rotational axis forms an angle of between 2° and 20° with a straight line that is parallel with the craniocaudal axis of a head of the examination object, said head being positioned at least partially within the second recess.

The craniocaudal axis of a head typically extends from the side of the head that faces the neck to the top of the head, e.g. centrally. Depending on the positioning of the head within the second recess relative to the first plane, the craniocaudal axis of a head can be so oriented as to be parallel with the first plane or tilted relative thereto. According to this embodiment variant, the first recess is therefore designed in such a way that the vibration generator can be tilted relative to the head of the examination object that is to be positioned. The rotational axis typically encloses an angle of any suitable range, such as e.g. between 3° and 10°, between 4° and 8°, 5°, etc., with a straight line that is parallel with the craniocaudal axis. Owing to the anatomy of a head which is positioned in the second recess, this allows particularly good penetration of the mechanical waves in the head, e.g. as a result of a potentially particularly large contact surface between head and vibration generator.

An embodiment variant of the elastography apparatus provides for the drive axis and/or the rotational axis to be parallel with a linear connection between a caudal-anterior position and a cranial-posterior position of the head that is to be positioned. In an embodiment, the first recess and the second recess are so shaped as to allow such an arrangement of the vibration generator relative to a head. According to this embodiment variant, the vibration generator is located in such a way that the rotational axis runs parallel with a straight line which is tilted relative to the craniocaudal axis in the direction of a linear connection between the chin and the rear of the head. Owing to the anatomy of a head which is positioned in the second recess, this allows particularly good penetration of the mechanical waves in the head, e.g. as a result of a potentially particularly large contact surface between head and vibration generator.

An embodiment variant of the elastography apparatus provides for the vibration generator to comprise a flexible rotating lead which is designed to connect the rotatable shaft to a step motor and/or to a transmit a rotation from a step motor to the rotatable shaft. The flexible rotating lead is typically pliable. The step motor can be implemented in accordance with any suitable design, such as that described in accordance with US20230296708A1, for example. This embodiment variant allows the vibration generator to be activated in a flexible manner.

An embodiment variant of the elastography apparatus provides for the flexible rotating lead to comprise a connection to the rotatable shaft and for said connection to comprise a bayonet fastener. Alternatively or additionally, the connection can comprise a locking nut. A bayonet fastener can typically be detached in a reversible manner, and therefore allows a resilient and flexible connection between the vibration generator and step motor.

An embodiment variant of the elastography apparatus provides for the first support element to be designed as a surface element in a first plane, and for that end of the rotatable shaft which faces towards the flexible rotating lead to be arranged, in a horizontal view of the first plane, above that end of the rotatable shaft which faces away from the flexible rotating lead. According to this embodiment variant, when the examination object is positioned horizontally in such a way that the head is arranged within the second recess, that end of the rotational axis and/or of the drive axis which faces towards the chin and/or the shoulders is therefore arranged above that end of the rotational axis and/or of the drive axis which faces towards the top of the head. That end of the drive axis of the rotatable shaft which faces the chin and/or the shoulders is typically arranged for a connection to the flexible rotating lead. This means that the flexible rotating lead can be arranged on the first support clement and/or partially integrated in the first support element and can be accessed in a particularly ergonomic manner, even if a head is already arranged in the second recess. This embodiment variant allows a particularly simple connection between the flexible rotating lead and the rotatable shaft.

An embodiment variant of the elastography apparatus provides for the elastography apparatus additionally to comprise a second support element. The second support element is so designed as to be positioned on a patient locating apparatus to assist in horizontally locating at least an upper body of the examination object, the second support element having a third recess, which is designed to accommodate at least part of the flexible rotating lead.

The second support element can comprise plastic and/or foam, for example. The third recess may e.g. be arranged on a first side, e.g. on the upper side of the second support element. The upper side of the second support element is characterized in that at least an upper body of the examination object can be positioned on the upper side. The third recess can also penetrate the full height of the second support element. The step motor is typically arranged outside the magnetic resonance device and/or the patient receiving region and/or the detector unit. The radiofrequency receiving coil unit, the first support element, and the vibration generator are typically arranged within the patient receiving region in the case of an MRE examination of the examination object. The flexible rotating lead allows impulses that are generated by the step motor to be transmitted and/or transferred to the vibration generator, i.e. into the patient receiving region. The third recess allows defined positioning and a defined course of the flexible rotating lead. The flexible rotating lead may e.g. be positioned flexibly in the third recess and outside the environment of the elastography apparatus. The at least partial locating of the flexible rotating lead in the third recess allows an examination object to be comfortably positioned on the second support element, and ensures a stable course of the flexible rotating lead during operation, making it possible to reduce and partially absorb vibrations. Moreover, the flexible rotating lead and the vibration generator are separated from the examination object in such a way that damage to these components, e.g. due to a weight and/or a weight shift of the examination object, can be avoided.

An embodiment variant of the elastography apparatus provides for the elastography apparatus to include the step motor. The step motor is typically connected to a control unit of the radiofrequency receiving coil unit and/or of the magnetic resonance device. This allows the elastography apparatus to be activated, e.g. in a manner that is synchronized with the magnetic resonance device.

An embodiment variant of the elastography apparatus provides for the elastography apparatus additionally to comprise a cushion which is arranged on the first plane between the first recess and the second recess. In an embodiment, the cushion can be or become arranged between the vibration generator and a head that is to be positioned in the second recess. A further hollow of the first support element can be provided between first recess and second recess, it being possible to position the cushion in said further hollow. The cushion can be a foam element and/or a feather cushion. The cushion is typically designed as a compressible cushion and/or gel cushion. The cushion typically comprises a cover which encloses a medium. The cushion, e.g. the cover, may e.g. be shaped in a flexible manner. The cushion is typically flat and/or has a height of less than any suitable dimension, such as e.g. less than 3 cm, less than 2 cm, less than 1 cm, etc. The medium can comprise a gas, e.g. air, or a liquid such as water, for example, or a gel. Such a cushion increases the comfort of the examination object, since it is thereby possible to avoid contact between the vibration generator, which is typically rigid, and the head. The cushion also allows particularly good transmission of the mechanical waves that can be generated by the vibration generator.

The disclosure further relates to a receiving unit comprising a radiofrequency receiving coil unit and an elastography apparatus according to the disclosure. The radiofrequency receiving coil unit is designed to receive magnetic resonance signals from the head of an examination object, and the elastography apparatus is arranged at least partially within the radiofrequency receiving coil unit. The radiofrequency receiving coil unit is typically designed as a head coil and/or a combined neck/head coil. The radiofrequency receiving coil unit is typically designed to at least partially surround the head of the examination object. The radiofrequency receiving coil unit may comprise any suitable number of receive channels, such as e.g. between 16 and 30 receive channels, 20 receive channels, etc. In an embodiment, the first support element is arranged at least partially within the radiofrequency receiving coil unit. The first support element need not have a connection to the radiofrequency receiving coil unit, e.g. does not have a permanent connection to the radiofrequency receiving coil unit. The first support element can be secured within the radiofrequency receiving coil unit in a reversible and/or removable manner.

The disclosure further relates to a magnetic resonance device comprising a detector unit and an elastography apparatus according to the disclosure and/or a receiving unit according to the disclosure. The detector unit typically comprises a main magnet, a radiofrequency antenna unit and a gradient coil unit, and is designed to generate MR signals. The magnetic resonance device is designed for MR elastography of the head. The magnetic resonance device may be designed for synchronized activation of the elastography apparatus and the detector unit.

Embodiment variants of the receiving unit and of the magnetic resonance device are designed in a similar manner to the embodiment variants of the elastography apparatus. The advantages of the receiving unit and of the magnetic resonance device correspond essentially to the advantages of the elastography apparatus, these being explained in detail above. Features, advantages or alternative embodiment variants cited in this context can also be applied in exactly the same way to the other claimed subject matter and vice versa.

### BRIEF DESCRIPTION OF THE DRAWINGS

Further advantages, features and details of the disclosure are derived from the exemplary embodiments described in the following and with reference to the drawings, in which:

FIG. 1 illustrates an embodiment of an example elastography apparatus, in accordance with the disclosure;

FIG. 2 illustrates an embodiment variant of an example vibration generator, in accordance with the disclosure;

FIG. 3 illustrates a position of an example vibration generator relative to a first support element in a first view, in accordance with the disclosure;

FIG. 4 illustrates a position of an example vibration generator relative to the first support element in a second view, in accordance with the disclosure;

FIG. 5 illustrates an example of a head and a vibration generator positioned on the first support element in a first view, in accordance with the disclosure;

FIG. 6 illustrates an example of a head and a vibration generator positioned on the first support element in a second view, in accordance with the disclosure; and

FIG. 7 illustrates an embodiment variant of an example magnetic resonance device with a receiving unit, in accordance with the disclosure.

### DETAILED DESCRIPTION OF THE DISCLOSURE

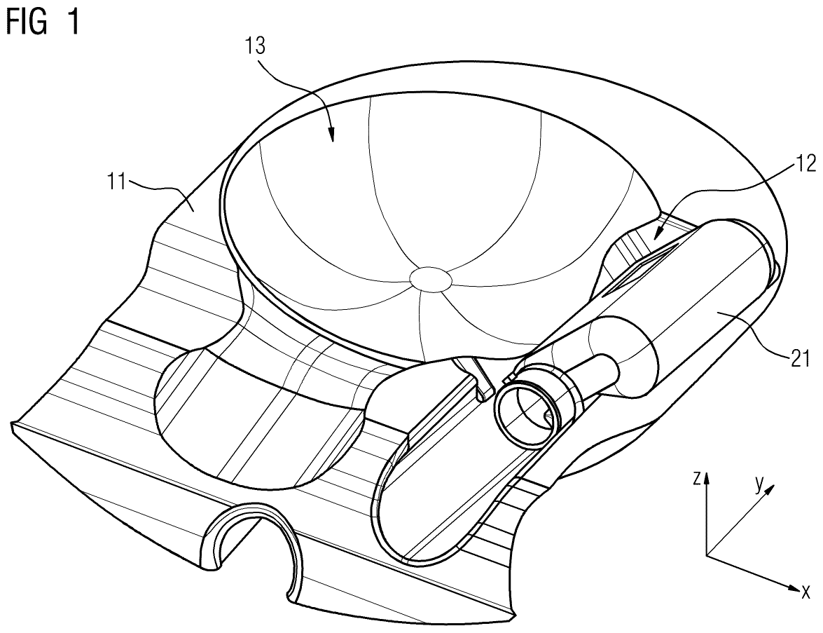

FIG. 1 illustrates an embodiment of an example elastography apparatus, in accordance with the disclosure. The illustrated elastography apparatus is designed to assist in magnetic resonance elastography of the head of an examination object 17. The elastography apparatus comprises a first support element 11, which can be positioned within a radiofrequency receiving coil unit 19 (also referred to herein as a receiving coil assembly; details not shown). The elastography apparatus also comprises a vibration generator 21, which is designed to generate mechanical waves. The first support element 11 has a first recess 12 for accommodating the vibration generator 21. In addition to this, the first support element 11 has a second recess 13, which is shaped in such a way that a head can be positioned at least partially therein. The first recess 12 is so shaped as to at least partially enclose the vibration generator 21 in a custom-fit manner. The vibration generator 21 is arranged at least partially within the first recess 12. In this case, the vibration generator 21 can be reversibly and removably positioned in the first recess 12, so that it can be arranged therein and withdrawn as required. The first support element 11 may be designed as a surface element, leaving aside the first recess 12 and the second recess 13. In this case, the position of the surface element can be approximated by a first plane, which corresponds to the x-y plane in the illustrated example.

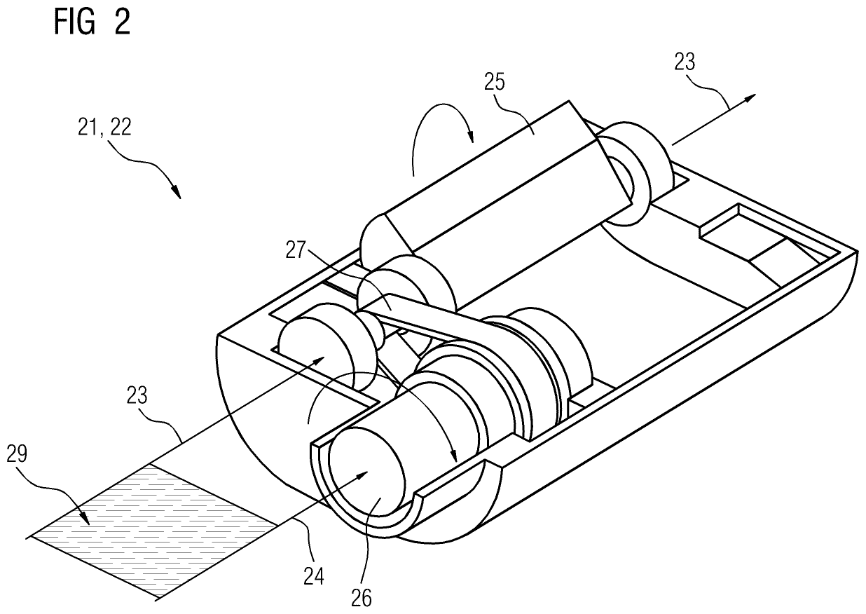

FIG. 2 illustrates an embodiment variant of an example vibration generator, in accordance with the disclosure. The vibration generator 21 is designed as a gravitational vibration generator 22 according to the illustrated embodiment variant. The gravitational vibration generator 22 comprises an eccentric mass 25 which can rotate about a rotational axis 23. In addition to this, the gravitational vibration generator 22 has a drive axis 24 which is located parallel with the rotational axis 23 and has a rotatable shaft 26. The rotatable shaft 26 is designed to drive the rotational axis 23 and therefore to drive the eccentric mass 25. To this end, the drive axis 24 is connected to the rotational axis 23 via a belt 27. The drive axis 24 and the rotational axis form a drive plane 29.

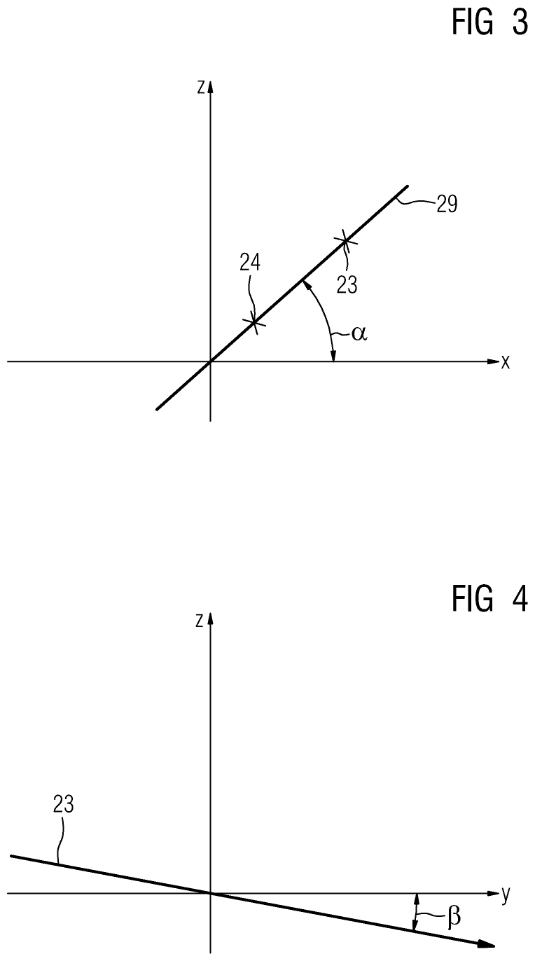

FIG. 3 illustrates a position of an example vibration generator relative to a first support element in a first view, in accordance with the disclosure. FIG. 3 shows a position of a vibration generator 21 relative to the first support element 11 in a first view. The system of coordinates here corresponds to the system of coordinates used in FIG. 1. According to this embodiment variant, the vibration generator 21 illustrated in FIG. 2 is positioned at least partially within the first recess 12 in such a way that the drive plane 29 encloses an angle α of that is between any suitable range of values, such as e.g. between 25° and 65°, an angle α of 45° in the illustrated example, etc., with the first plane, i.e. the x-y plane. The rotational axis 23 here is further away from the first plane, i.e. the x-y plane, than the drive axis 24.

FIG. 4 illustrates a position of an example vibration generator relative to the first support element in a second view, in accordance with the disclosure. FIG. 4 shows a position of a vibration generator 21 relative to the first support element 11 in a second view. The second view is perpendicular to the first view used in FIG. 3, and the system of coordinates used in FIG. 4 corresponds to the system of coordinates used in FIG. 1. According to this embodiment variant, the vibration generator 21 illustrated in FIG. 2 is positioned at least partially within the first recess 12 in such a way that the rotational axis 23, and hence the drive axis 24 likewise, encloses an angle β of between any suitable angular range, such as e.g. between 2° and 20°, an angle β of 10° in the illustrated example, etc., with the first plane, i.e. the x-y plane. The craniocaudal axis of the head can lie within the first plane, i.e. the x-y plane. The craniocaudal axis of the head can have precisely one intersection point with the first plane, i.e. the x-y plane. According to this embodiment variant, the drive axis 24 and/or the rotational axis 23 of the vibration generator 21, 22 can form an angle of any suitable range such as e.g. between 2° and 20° with a straight line that is parallel with the craniocaudal axis of a head of the examination object 17, which head is to be positioned at least partially within the second recess. In the illustrated case, the drive axis 24 and the rotational axis 23 are moreover parallel with a linear connection between a caudal-anterior position and a cranial-posterior position of the head that is to be positioned. The drive axis 24 and the rotational axis 23 are therefore tilted relative to the first plane by at most 20° towards the chin of the head.

The first recess 12 may e.g. be embodied in such a way that that end of the vibration generator 21 which faces towards the chin of the head that is to be positioned is arranged on the same side of the first plane, i.e. the x-y plane, as the chin and/or has the same operational sign as the chin or the nose of the head along the z-axis. The first recess 12 is may e.g. be embodied in such a way that that end of the vibration generator 21, which faces towards the cranial end of the head is arranged on the opposite side of the first plane, i.e. the x-y plane, to the chin and/or has the opposite operational sign to the chin and/or nose of the head along the z-axis.

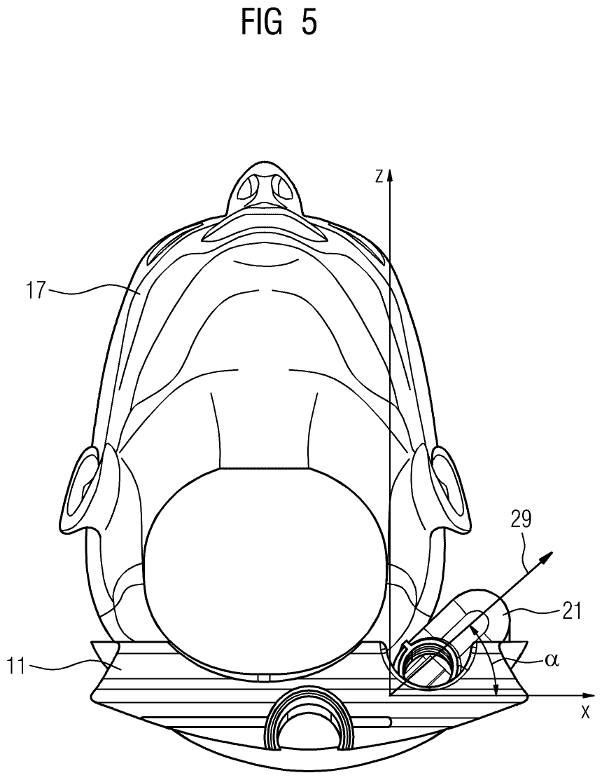

FIG. 5 illustrates an example of a head and a vibration generator positioned on the first support element in a first view, in accordance with the disclosure. FIG. 5 shows a head of the examination object 17, positioned on the first support element 11, and a vibration generator 21 in a first view in a schematic illustration which is analogous to FIG. 3.

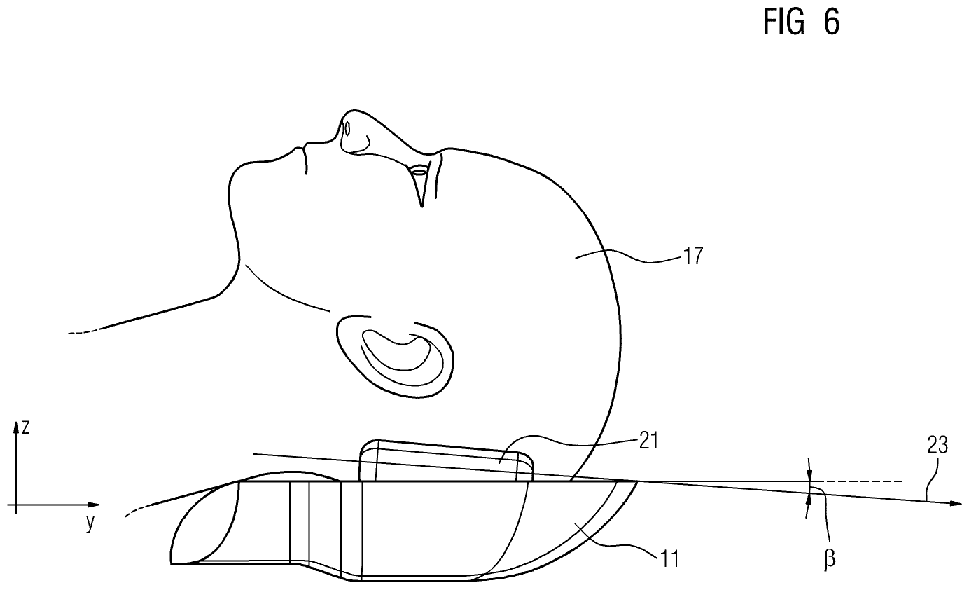

FIG. 6 illustrates an example of a head and a vibration generator positioned on the first support element in a second view, in accordance with the disclosure. FIG. 6 shows a head of the examination object 17, positioned on the first support element 11, and a vibration generator 21 in a second view in a schematic illustration which is analogous to FIG. 4.

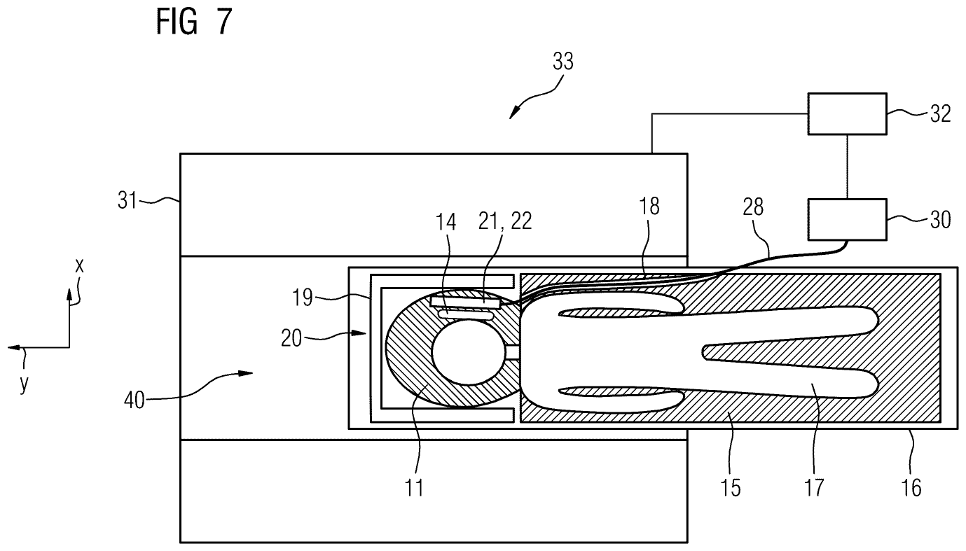

FIG. 7 illustrates an embodiment variant of an example magnetic resonance device with a receiving unit, in accordance with the disclosure. FIG. 7 shows an embodiment variant of a magnetic resonance device 33 with an embodiment variant of a receiving unit 20 in a schematic illustration. According to this embodiment variant, the magnetic resonance device 33 comprises a hollow cylindrical detector unit 31 (also referred to herein as a detector) enclosing, e.g. concentrically surrounding, a cylindrical patient receiving region 40. The cylindrical patient receiving region 40 is designed to accommodate an examination object 17. The longitudinal axis of the cylindrical patient receiving region 40 corresponds to the y-axis according to FIG. 1. The examination object 17 can be pushed into the patient receiving region 40 by means of a patient locating apparatus 16 of the magnetic resonance device 33. The detector unit 31 typically comprises a main magnet (details not shown), a gradient coil unit (details not shown) and/or a radiofrequency antenna unit (details not shown), which is designed to transmit excitation pulses.

For the purpose of controlling the detector unit 31, the magnetic resonance device 33 has a control unit 32 (Also referred to herein as a controller). The control unit 32 controls the magnetic resonance device 33 centrally, carrying out MR control sequences, for example. In addition to this, the control unit 32 comprises a reconstruction unit (details not shown) for reconstruction of medical image data. The control unit 32 can have a display unit (details not shown) and an input unit (details not shown). In addition to this, the control unit 32 can be designed to evaluate MRE data.

The receiving unit 20 comprises a radiofrequency receiving coil unit 19 (also referred to herein as an RF receiving coil assembly or an RF receiver) which is designed to receive magnetic resonance signals from a head of an examination object 17. The radiofrequency receiving coil unit 19 in the illustrated embodiment variant is designed as a head coil, which is designed to at least partially surround the head and/or the neck of an examination object 17 in the manner of a cage. The radiofrequency receiving coil unit 19 may have a plurality of receiving coils and/or receive channels.

In addition to this, the receiving unit 20 comprises an elastography apparatus, which is arranged at least partially within the radiofrequency receiving coil unit 19. The embodiment variant of the elastography apparatus as illustrated in FIG. 5 comprises a vibration generator 21 that is designed as a gravitational vibration generator 22 with a flexible rotating lead 28. The flexible rotating lead 28 is designed to connect the rotatable shaft 26 to a step motor 30 and to transmit a rotation from a step motor 30 to the rotatable shaft 26. For this purpose, the flexible rotating lead 28 has a connection to the rotatable shaft 26, said connection comprising a bayonet fastener (details not shown). According to this embodiment variant, the step motor 30 is included in the elastography apparatus. In addition to this, the step motor 30 has a connection to the control unit 32. The control unit 32 can be designed to control the elastography apparatus. The control unit 32 may e.g. be designed to activate the step motor 30 and/or to synchronize the step motor 30 with an MR control sequence that is to be output by the detector unit 31. In addition to this, the control unit 32 can be designed to evaluate the magnetic resonance signals received from the radiofrequency receiving coil unit 19, with reference to information relating to the activation of the elastography apparatus.

According to this embodiment variant, the elastography apparatus additionally comprises a second support element 15, which is designed to be positioned on the patient locating apparatus 16 of the magnetic resonance device 33. In this case, the patient locating apparatus 16 is designed to assist in horizontally locating at least an upper body of the examination object 17, and the second support element 15 can be positioned between the patient locating apparatus 16 and a prone examination object 17. The second support element 15 has a third recess 18, which third recess 18 is designed to accommodate at least part of the flexible rotating lead 28. In addition to this, the elastography apparatus according to this embodiment variant comprises a cushion 14, which can be arranged between the first recess 12 and the second recess 13, e.g. between the vibration generator 21 and a head that is to be positioned.

The illustrated magnetic resonance device 33 can of course comprise further components which are customarily included in magnetic resonance devices 33. The way in which a magnetic resonance device 33 generally functions is known to a person skilled in the art and therefore a detailed description of the further components has been omitted.

Although the disclosure is illustrated and described in detail by means of the exemplary embodiments, the disclosure is not restricted by the examples disclosed and other variations may be derived therefrom by a person skilled in the art without departing from the scope of the disclosure.

The various components described herein may be referred to as “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such units, as applicable and relevant, may alternatively be referred to herein as “circuitry,” “controllers,” “processors,” or “processing circuitry,” or alternatively as noted herein.

What is claimed is:

1. An elastography apparatus configured to assist in magnetic resonance elastography of a head of an examination object, comprising:
a first support element configured to be positioned within a radiofrequency (RF) receiving coil assembly; and
a vibration generator configured to generate mechanical waves,
wherein the first support element comprises a first recess for receiving the vibration generator,
wherein the first support element comprises a second recess for receiving the head, and
wherein the first recess is shaped to at least partially enclose the vibration generator, the vibration generator being arranged at least partially within the first recess.

2. The elastography apparatus as claimed in claim 1, wherein the vibration generator is reversibly and removably positioned within the first recess.

3. The elastography apparatus as claimed claim 1, wherein the vibration generator comprises:
a gravitational vibration generator having an eccentric mass that rotates about a rotational axis; and
a rotatable shaft that rotates about a drive axis that is located parallel with the rotational axis,
wherein the rotatable shaft is configured to drive the eccentric mass, and
wherein the drive axis and the rotational axis form a drive plane.

4. The elastography apparatus as claimed in claim 3, wherein the first support element comprises a surface element in a first plane, and
wherein the first recess is configured such that, when the vibration generator is positioned at least partially within the first recess, the drive plane encloses an angle between 25° and 65° with the first plane.

5. The elastography apparatus as claimed in claim 4, wherein the drive axis is closer to the first plane than the rotational axis.

6. The elastography apparatus as claimed in claim 3, wherein the first support element comprises a surface element in a first plane, and the vibration generator comprises a housing assembly that externally seals components included in the gravitational vibration generator,
wherein the housing assembly comprises at least one flat surface, and
wherein, when the vibration generator is positioned at least partially within the first recess, the flat surface encloses an angle between 25° and 65° with the first plane.

7. The elastography apparatus as claimed in claim 3, wherein the first support element comprises a surface element in a first plane, and
wherein, when the vibration generator is positioned at least partially within the first recess, the drive axis and/or the rotational axis forms an angle of between 2° and 20° with the first plane.

8. The elastography apparatus as claimed in claim 3, wherein the vibration generator comprises a flexible rotating lead configured to connect the rotatable shaft to a step motor and/or to transfer a rotation from a step motor to the rotatable shaft.

9. The elastography apparatus as claimed in claim 8, wherein the first support element comprises a surface element in a first plane, and
wherein an end of the rotatable shaft facing towards the flexible rotating lead is arranged, in a horizontal view of the first plane, above an end of the rotatable shaft that faces away from the flexible rotating lead.

10. The elastography apparatus as claimed in claim 8, wherein the flexible rotating lead comprises a bayonet fastener connection to the rotatable shaft.

11. The elastography apparatus as claimed in claim 1, further comprising:
a step motor.

12. The elastography apparatus as claimed in claim 8, further comprising:
a second support element configured as to be positioned on a patient locating apparatus to assist in horizontally locating at least an upper body of the examination object,
wherein the second support element comprises a third recess configured to accommodate at least part of the flexible rotating lead.

13. The elastography apparatus as claimed in claim 1, further comprising:
a cushion arranged on a first plane between the first recess and the second recess.

14. A receiver, comprising:
a radiofrequency (RF) receiving coil assembly configured to receive magnetic resonance signals from a head of an examination object; and
an elastography apparatus configured to assist in magnetic resonance elastography of the head of the examination object, the elastography apparatus comprising:
a first support element configured to be positioned within a radiofrequency (RF) receiving coil assembly; and
a vibration generator configured to generate mechanical waves,
wherein the first support element comprises a first recess for receiving the vibration generator,
wherein the first support element comprises a second recess for receiving the head,
wherein the first recess is shaped as to at least partially enclose the vibration generator, the vibration generator being arranged at least partially within the first recess, and
wherein the elastography apparatus is arranged at least partially within the RF receiving coil assembly.

15. A magnetic resonance device, comprising:
a detector; and
an elastography apparatus configured to assist in magnetic resonance elastography of a head of an examination object, the elastography apparatus comprising:
a first support element configured to be positioned within a radiofrequency (RF) receiving coil assembly; and
a vibration generator configured to generate mechanical waves,
wherein the first support element comprises a first recess for receiving the vibration generator,
wherein the first support element comprises a second recess for receiving the head, and
wherein the first recess is shaped as to at least partially enclose the vibration generator, the vibration generator being arranged at least partially within the first recess.
